(19) United States
(12) Patent Application Publication
(10) Pub. No.: US 2020/0121410 A1
(43) Pub. Date: Apr. 23, 2020

(54) IMPROVEMENTS IN OR RELATING TO PACKAGING

(71) Applicant: Herhealth Limited, London (GB)

(72) Inventor: Hazem El-Refaey, London (GB)

(21) Appl. No.: 16/610,781

(22) PCT Filed: May 3, 2018
(86) PCT No.: PCT/EP2018/061304

§ 371 Date: Nov. 4, 2019

(30) Foreign Application Priority Data
May 4, 2017 (GB) 1707145.7

(51) Int. Cl. A61B 50/30; A61B 50/33; B65D 43/02; B65D 55/06; B65D 1/36; B65D 51/20

(52) U.S. Cl. CPC A61B 50/30; A61B 50/33; B65D 43/0235; A61B 2017/0023; B65D 1/36; B65D 51/20; B65D 55/06

(57) ABSTRACT

The invention provides a sealed pack which pack comprises a container and a lid wherein the container has an internal tamper-evident seal and the lid has an external tamper-evident seal; wherein the internal seal is arranged between the container and the lid; and wherein the lid is formed from a resilient material such that it protects the container. In one embodiment, the invention provides a pack of surgical instruments for use in a pre-determined surgical intervention which pack comprises one or more sterilised surgical instruments suitable for use in the pre-determined surgical intervention and a tray for receiving the one or more sterilised surgical instruments wherein the pack is sealed by at least two seals.

The present invention relates to packaging for the secure transport of items.

There is a need to transport items securely such that it can be checked at each stage of their journey that there has been no tampering. For example, sterile surgical instruments need to be transported securely to the point of use such that it is clear that they are still sterile, food needs to be transported securely to its consumer such that it is clear that the food is still sealed at the point of use and high value items need to be transported securely such that it is clear at the point of receipt by their owner that there has been no tampering.

Current packaging does not provide means for checking that the contents of a package are secure at each stage of its journey.

A way of ameliorating these problems has been sought.

According to the invention there is provided a sealed pack which pack comprises a container and a lid wherein the container has an internal tamper-evident seal and the lid has an external tamper-evident seal; wherein the internal seal is arranged between the container and the lid; and wherein the lid is formed from a resilient material such that it protects the container.

Advantages of the invention include that the security of the contents of the pack may be checked in different ways during transportation of the pack. During transit, the security of the pack may be checked by inspecting the lid and its external seal. After removal of the lid and before use or otherwise opening of the container, its internal seal may be checked by a user of the container to ensure that its contents have not been tampered with.

In some embodiments, the container may be a tray, bag or pouch. In some embodiments, the pack may comprise a support for protecting the container, particularly where the container is a bag or a pouch. In some embodiments, the container may have a collar to allow it to be suspended in a support.

In some embodiments, the lid, container and/or the internal tamper-evident seal may be transparent such that the contents of the container may be checked before use, for example before a tamper-evident seal is opened.

In some embodiments, the lid may have a clip to secure the lid to the container. In some embodiments, the external tamper-evident seal may be provided on the clip. In some embodiments, the external tamper-evident seal may be a film seal. Such a film seal may be formed from a layer of plastics material which is wrapped around the pack such that the lid cannot be opened without its removal.

In some embodiments, the pack may be a pack of sterilised surgical instruments for use in a pre-determined surgical intervention which pack comprises a tray, a lid to protect the tray and one or more surgical instruments suitable for use in the pre-determined surgical intervention wherein the pack is sealed by an external seal and an internal seal.

Surgical theatres carry out a large number of different interventions. Some of these interventions can be scheduled and planned well in advance. However, other interventions need to be carried out with short notice when even a minor delay can impact severely on the likelihood of a successful outcome. Before an intervention can start, a number of steps need to be taken such as the assembly of the right personnel in a surgical theatre with the correct equipment and suitable instruments. Typically a further step is the sterilisation of the surgical instruments.

In most hospitals at least in the UK, there is a central sterilisation store (CCST) which provides streamlined sterilisation services for all specialities but they can be overwhelmed. It is not unknown for them to make mistakes in the preparation of the instruments. The staff working in these stores are not medical and they have no knowledge of surgical procedures. It is therefore also not unknown that some procedures in specialities like orthopaedics have to be cancelled due to the absence of a small but crucial instrument in the provided surgical trays. Central sterilisation stores can be overwhelmed with work and over the years several changes occurred in the way these important places provide their services.

The preparation of the instruments prior to surgery is a well-established routine. The metal trays provided by the CSST are opened in a dedicated area or in a side room attached to a surgical theatre. Nursing staffs that have specific training in the handling of these surgical instruments undertake the preparation, almost out of sight, prior to surgery.

The preparation process has two aims. Firstly to ensure that all required instruments are available and secondly to document that the number of the instruments that will be counted at the end of the procedure is the same as the number counted at the beginning. This process can be time consuming and introduces potential risks into the procedure.

Advantages of the invention include that one possible cause of delay in emergency interventions is removed because a surgical theatre may be provided with one or more packs according to the invention. There is no need for the staff to sterilise or prepare the surgical instruments as the instruments in the pack are already sterilised. The only action which needs to be taken is to check that at least one of the seals on the pack is intact.

In some embodiments, the tray may form one or more tray compartments for receiving the one or more surgical instruments. Advantages of having one or more tray compartments include that the instruments may be arranged in the tray compartments in a way that will fit with the logic of the operation.

Herein it should be understood that a tamper-proof seal is a seal which cannot be opened without an element of the seal being destroyed such that it is clear that there is evidence of tampering. In some embodiments, at least one of the seals may be a lid clip seal such that a seal is provided where the lid fastens on to the container or tray. In some embodiments, at least one of the seals may be transparent. In some embodiments, at least one of the seals may include a transparent inner seal. The advantage of having a transparent inner seal is that the lid of the pack may be removed prior to surgery such that the surgical instruments can be seen and counted without breaking the last seal. The pack having an inner seal which is still intact can be used within several hours of breaking the other seal(s).

Caesarean Section (CS) is an integral surgical intervention in obstetric care for pregnant and labouring women. The procedure has evolved over two hundred years with an incidence that has increased from less than 1% of deliveries to currently around 20%. The main indications for this intervention have ranged from a last desperate measure in the management of obstructed labour to an emergency procedure to save the foetal or maternal life. Caesarean sections for foetal distress or placental abruption or maternal haemorrhage are examples for these particular scenarios.

An emergency caesarean section occurs when there is an urgent need for performing this procedure without delay in certain circumstances. This urgency is not always reflected on the speed by which the procedure is initiated. The slow or protracted lag between decision-making and the time of initiating the operation is well recognised. This is generally seen as an inevitable delay. Health providers are urged to perform these emergency procedures within 30 minutes. Professional bodies use 30 minutes as a medical or legal parameter to measure the quality of patient care in an emergency situation. A Caesarean Section commencing after 30 minutes from taking the decision i.e. knife to skin is considered to be a procedure that was performed urgently.

A lag of 30 minutes should be unjustifiable considering that most patients requiring this emergency procedure have epidural cannula already in site. This means that the delay is not necessarily a function of commencing anaesthesia. The vast majority of patients requiring this procedure have an epidural analgesia or spinal anaesthesia which is usually sufficient to perform the operation without delay.

There are many factors that contribute to this “unavoidable delay”. These involve factors such as moving the patient to the operating theatre and summoning skilled scrub nurses to theatre. However it is the preparation of the surgical instruments by those skilled scrub nurses that contributes mostly to this delay.

In some embodiments, the one or more surgical instruments may be single use or disposable surgical instruments. In some embodiments, the one or more surgical instruments may be made of a plastic material and/or metal material (such as tungsten, graphite, titanium and/or aluminium), for example recyclable aluminium. Advantages of the single use surgical instruments include that it provides uniformity in the preparation of the surgical instruments prior to surgery and so reduces delays in preparing the instruments, cleaning them, disposing them. Such uniformity is a desirable feature that will lead to streamlining of service, minimising of risk, and economising efforts and time, for the following reasons:
Previously most interventions have used a combination of both, for example in CS almost all the instruments are reusable and made of stainless steel. Some disposable items are provided in an open or unsealed state and are added to the set of the standard reusable instruments. As an example in CS and in any other similar surgery which require suction for removal of a fluid, a suction tube is provided separately as a disposable item. The reason is that suction tubes or cords cannot be cleaned adequately in a central sterilisation store. The cord would need personnel to handle it and direct water jet inside it to clean it. Most instruments today are cleaned by immersion in cleaning solutions and machines which cannot clean a tube of such nature. This kind of tubing is used in abortion to suck products of conception and Laparotomy or intra-abdominal operations to suck blood. In Caesarean section a suction tube is essential to suck the gushing amniotic fluid when the uterine cavity is incised;
Another example is laparoscopic surgery where many disposable instruments exist but they never cover the whole range of the operation. Reusable instruments are added in the form of scissors, polar diathermy etc;
The increasing awareness of the transmission of blood borne diseases like hepatitis B, C and CJD increases the need for extra vigilance required with regard to sterilising surgical instruments and, with the potential cross infectivity between patients, despite sterilisation. This emphasized the importance of the concept of traceability of reusable instruments. Complicated systems and software based on bar codes have been set to enable tracing the multiple use of any surgical instrument. This has added cost which could be avoided by making the surgical instruments disposable.
There have been a number of barriers to changing the approach of preparing the instruments for surgical use. This is so since the instruments have to be sterilised and has to come from a central sterilising store. Any changes in the nature of instruments remained patchy or specific to one or two instruments. Focussing primarily on the improvement of the technicality of the use of the instruments rather than on its handling. Single use instruments are essential to bypass these problems since they will bypass the role of the CSST.

In some embodiments, the pack may comprise a tray. In some embodiments, the tray may comprise one or more tray compartments for receiving the one or more surgical instruments. In some embodiments, the tray compartments may be in the form of grooves or slots. In some embodiments, the one or more tray compartments may be colour coded according to the purpose or use of the one or more surgical instruments. The advantages of colour coding the surgical instruments or their tray compartments is that it helps the surgical theatre support staff with their handling of the surgical instruments and enables less well trained support staff to assist in the intervention, for the following reasons:
During surgery, the surgeon is not allowed to handle or pick the surgical instruments from the tray. The presence of a trained scrub nurse with training and exposure to the drills of actions during an intervention (such as CS) is thus necessary. Scrub nurses should be familiar with what is happening during the surgery and the anatomy of the relevant parts of the body (such as the abdomen, uterus and human pelvis in CS). The scrub nurse should be capable to predict and expect the steps of the operation and anticipate the surgeon's demand for a surgical instrument and to hand him the required surgical instrument(s) prior to him asking for it. He/she should be to spontaneously hand the surgical instruments to the surgeon depending on the step or the layer of tissues he/she is handling. It is part of the training of surgical scrub nurses to be able to play this role in operating theatres. Lately, and due to cost effectiveness, midwives (in the case of CS) are sometimes asked to play the role of the scrub nurse in theatre. They are usually given a summary and speedy training to enable them to provide this activity. They are necessarily therefore less familiar with these drills or the instruments. Simplifying the role of the scrub nurse and/or midwives in handling the appropriate instrument is thus beneficiary.
No concerted effort was ever made to create a pack or a tray of instruments, with inherent anatomical logic, for a specific use and to cover the needs of a surgeon from A to Z in a simple structured approach. A revolutionary effort is required to speed up the handling procedure. This can only be achieved with a pack that contains all the important instruments that are colour coded such that they can easily be counted before and after the procedure.

In some embodiments, the pack according to the invention may include the majority of surgical instruments required for the pre-determined surgical intervention. For example, the pack may contain from 50%, for example from 55%, 60%, 65% or 70% to 75%, 80%, 85%, 90%, 95% or 99% of the required surgical instruments. The advantages of providing a pack containing the majority of surgical instruments required for the pre-determined surgical intervention include that preparation process for the intervention may be streamlined as the counting of the surgical instruments takes less time and that the following problems are ameliorated:
The tray of CS surgical instruments provided to labour wards is generally over-packed with instruments and this can create problems. The reason for this is to accommodate for variations in techniques and to suit the different needs of different surgeons. This is historical and has not been addressed seriously or methodologically. A pack containing 60% of these instruments will suffice in 95% of cases and will enable the streamlining of the process;
The instruments are sterilised in the central sterilisation store (CSST). They are usually brought to labour wards in metal trays, which contains all the “required” instruments. In total there are 54 instruments as shown in Table 1. The number of instruments and/or types may vary between one hospital and another. The surgical instruments used in CS may safely be reduced to the set of instruments shown in Table 2 below, resulting in increased efficiency for the surgeon and for the use of the operating theatre.

TABLE 1
The current Caesarean section set of instruments

| | Number of instruments |
|---|---|
| Contents | included on the tray |
| Allis Tissue Forceps | 2 |
| Green Armitage Forceps Curved | 2 |
| Green Armitage Forceps Straight | 5 |
| Criles Artery Forceps Curved | 10 |
| Spencer Wells Artery Forceps 7″ | 5 |
| Straight | |
| Kocher Artery Forceps 8″ Straight | 2 |
| Mayo Needleholder 7″ | 2 |
| Cord Scissors | 1 |
| Dressing Scissors | 1 |
| Mayo Scissors Straight | 1 |
| Mayo Scissors Curved | 1 |
| McIndoe Scissors, Curved | 1 |
| Rampley Sponge Holding Forceps | 5 |
| Bonney Toothed Dissecting Forceps | 1 |
| Dissecting Forceps 7″ Non-Toothed | 1 |
| Waugh Dissecting Forceps Non-Toothed | 1 |
| Waugh Dissecting Forceps Toothed | 1 |
| Bard Parker Handle No. 4 | 2 |
| Wrigley Obstetric Forceps (Pair L & R) | 1 |
| Doyen Hand Retractor | 2 |
| Langenbeck Retractors Medium | 2 |
| Morris Retractors | 2 |
| Mono Polar Diathermy Cable | 1 |
| Towel Clip | 1 |
| Diathermy Dissecting Forceps 8″ | 1 |
| Total | 54 |

In some embodiments, the pack may contain one or more checklist which lists the names of the one or more surgical instruments in the pack. Before a surgical intervention starts, two of the auxiliary staff in the surgical theatre (such as nurses or midwives) will check the surgical instruments by counting them. One member of staff will loudly state the name of each instrument and another will mark that he/she agrees on the presence of each specific instrument. The checklist aids this process. This process is repeated three times. The pack may contain three checklists or a single checklist may have at least three checkboxes against each instrument name such that the check list may be usable at least three times. Generally speaking, all the instruments are, therefore, counted prior to the beginning of the procedure, after the closure of the patient's body and once more at the end of the procedure.

In some embodiments, the tray may be formed from a disposable, resilient and sterilisable material such as a plastics material. In some embodiments, the tray may have a lid. In some embodiments, the lid may be hinged to the tray. In some embodiments, the lid and/or tray may be formed from a resilient, sterilisable, disposable transparent material such as a transparent plastics material. Advantages of having a transparent lid and/or tray include that the nurses are able to count the surgical instruments without opening the pack prior to the start of the surgical intervention.

In some embodiments, the one or more surgical instruments may include one or more of the following surgical instruments: graspers (such as forceps); clamps and/or occluders (suitable for use with blood vessels and/or other organs); retractors (suitable for use in spreading open skin, ribs and/or other tissue); distractors, positioners and/or stereotactic devices; mechanical cutters (such as scalpels, lancets, drill bits, rasps, trocars, Ligasure, Harmonic scalpel, surgical scissors, water jet nozzles, rongeurs); dilators and/or specula (suitable for allowing access to narrow passages or incisions); suction tips and/or tubes (suitable for removal of bodily fluids); sealing devices (such as surgical staplers or electro-cauterisers); irrigation and/or injection needles including their tips and/or tubes (suitable for introducing fluid); powered devices (such as drills and/or dermatomes); scopes and/or probes (including endoscopes and/or tactile probes); carriers and appliers (suitable for use with optical, electronic and mechanical devices); tissue disruptors (such as ultrasound tissue disruptors, microtomes, cryotomes, cutting lasers); and/or measurement devices such as rulers and/or calipers.

In some embodiments, the pre-determined surgical intervention may be one or more of the following interventions: decompressive craniectomy, hemispherectomy, anterior temporal lobectomy, hypophysectomy, amygdalohippocampectomy, ventriculostomy, craniotomy, pallidotomy, thalamotomy, lobotomy, bilateral cingulotomy, cordotomy, rhizotomy, neurosurgery, psychosurgery, brain biopsy, ganglionectomy, sympathectomy/endoscopic thoracic sympathectomy, neurectomy, axotomy, vagotomy, nerve biopsy, endocrine, hypophysectomy, thyroidectomy, parathyroidectomy, adrenalectomy, pinealectomy, punctoplasty, trabeculoplasty, photorefractive keratectomy, trabeculectomy, iridectomy, vitrectomy, dacryocystorhinostomy, radial keratotomy, mini asymmetric radial keratotomy (M.A.R.K.), corneal transplantation, otoplasty, stapedectomy, mastoidectomy, auriculectomy, myringotomy, rhinoplasty, septoplasty, rhinectomy, laryngectomy, pneumonectomy, tracheostomy, sinusotomy, pneumotomy, cricothyroidotomy, cricothyrotomy, bronchotomy, thoracotomy, thyrotomy, tracheotomy, pleurodesis, lung transplantation, angioplasty, valvuloplasty, pericardiectomy, endarterectomy, cardiotomy, pericardiotomy, heart transplantation, tonsillectomy, adenoidectomy, thymectomy, splenectomy, lymphadenectomy, thymus transplantation, spleen transplantation, splenopexy, lymph node biopsy, uvulopalatoplasty, palatoplasty, gingivectomy, glossectomy, esophagectomy, gastrectomy, appendectomy, proctocolectomy, colectomy, hepatectomy, cholecystectomy, pancreatectomy, pancreaticoduodenectomy, gastrostomy (e.g. percutaneous endoscopic gastrostomy), gastroduodenostomy, gastroenterostomy, ileostomy, jejunostomy, colostomy, cholecystostomy, hepatoportoenterostomy, sigmoidostomy, uvulotomy, myotomy (Heller myotomy or pyloromyotomy), anal sphincterotomy, lateral internal sphincterotomy, vertical banded gastroplasty, gastropexy, colon resection, Nissen fundoplication, hernia repair, omentopexy, liver biopsy, urethroplasty, pyeloplasty, nephrectomy, cystectomy, nephrostomy, ureterostomy, cystostomy (e.g. suprapubic cystostomy), urostomy, nephrotomy, nephropexy, urethropexy, lithotripsy, kidney transplantation, renal biopsy, phalloplasty, scrotoplasty, vasectomy, penectomy, orchidectomy, prostatectomy, posthectomy, gonadectomy, vasovasostomy, vasoepididymostomy, meatotomy, circumcision, foreskin restoration, orchiopexy, prostate biopsy, vaginoplasty, clitoroplasty, labiaplasty, tuboplasty, fimbrioplasty, cervicectomy, clitoridectomy, oophorectomy, salpingoophorectomy, salpingectomy, hysterectomy, vaginectomy, vulvectomy, salpingostomy, amniotomy, clitoridotomy, hysterotomy, hymenotomy, episiotomy, symphysiotomy, tubal ligation, tubal reversal, colporrhaphy, caesarean section, hymenorrhaphy, endometrial biopsy, acromioplasty, khyphoplasty, mentoplasty, acromioplasty, arthroplasty, rotationplasty, ostectomy (e.g. femoral head ostectomy, vertebrectomy, coccygectomy or astragalectomy), corpectomy, facetectomy, laminectomy (hemilaminectomy), synovectomy, discectomy, osteotomy, arthrotomy, laminotomy, foraminotomy, epiphysiodesis, arthrodesis, arthroscopy, ulnar collateral ligament reconstruction, bursectomy, amputation (e.g. hemicorporectomy or hemipelvectomy), myotomy, tenotomy, fasciotomy, muscle biopsy, amputation, tendon transfer, mammoplasty, lumpectomy, mastectomy, breast implant, mastopexy, breast reconstruction, breast reduction plasty, V-plasty, VY-plasty, W-plasty, Z-plasty, escharotomy, skin biopsy, abdominoplasty, hernioplasty, frenuloplasty, Z-plasty, diverticulectomy, frenectomy, hemorrhoidectomy, mastoidectomy, thrombectomy, embolectomy, ganglionectomy, lobectomy, myomectomy, panniculectomy, ureterosigmoidostomy, fistulotomy, laparotomy, myringotomy, sphincterotomy, commissurotomy, abdominal surgery, inguinal hernia surgery, biopsy, brostrom procedure, cauterization, grafting, hypnosurgery, laparoscopy and/or Nuss procedure.

The invention will now be illustrated with reference to the following Figures of the accompanying drawings which are not intended to limit the scope of the claimed invention:

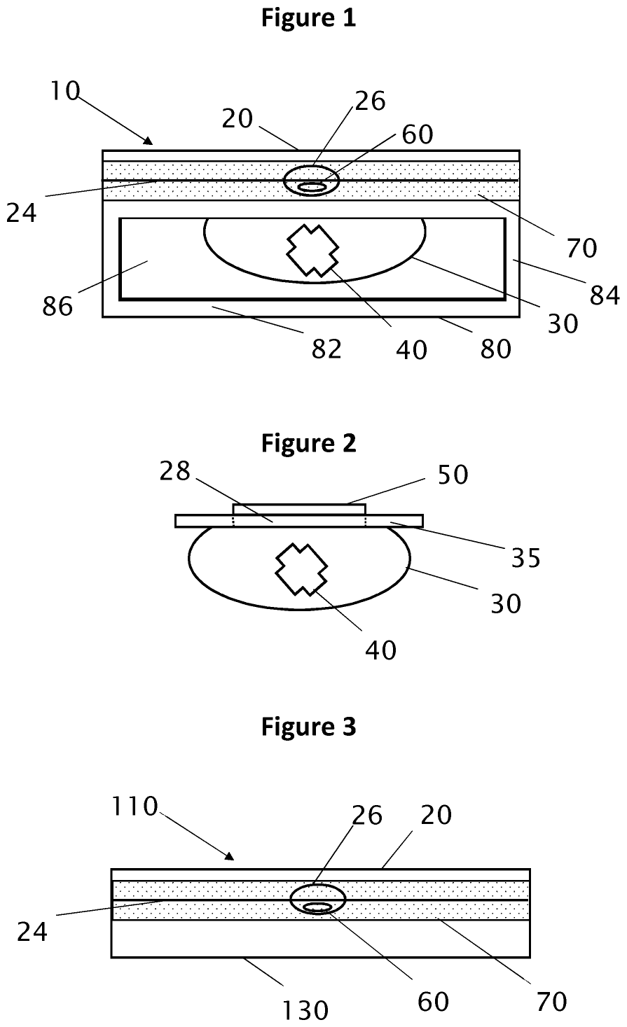

FIG. 1 shows a schematic front view of a sealed pack according to a first embodiment of the invention;

FIG. 2 shows a schematic view of a first embodiment of a container for use in the sealed pack according to the first embodiment of the invention;

FIG. 3 shows a schematic front view of a sealed pack according to a second embodiment of the invention;

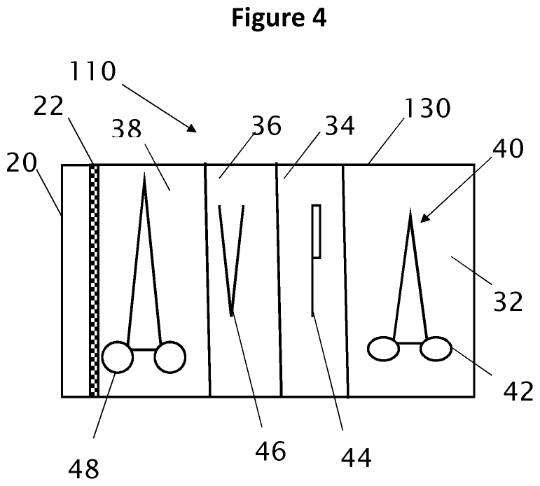

FIG. 4 shows a schematic plan view of a second embodiment of a container for use in the sealed pack according to the second embodiment of the invention;

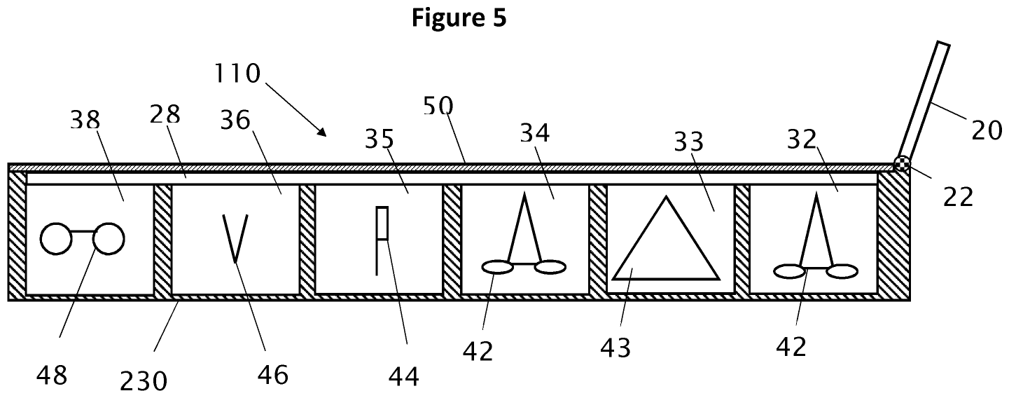

FIG. 5 shows a schematic cross-sectional view of the second embodiment of a container for use in the sealed pack according to the second embodiment of the invention;

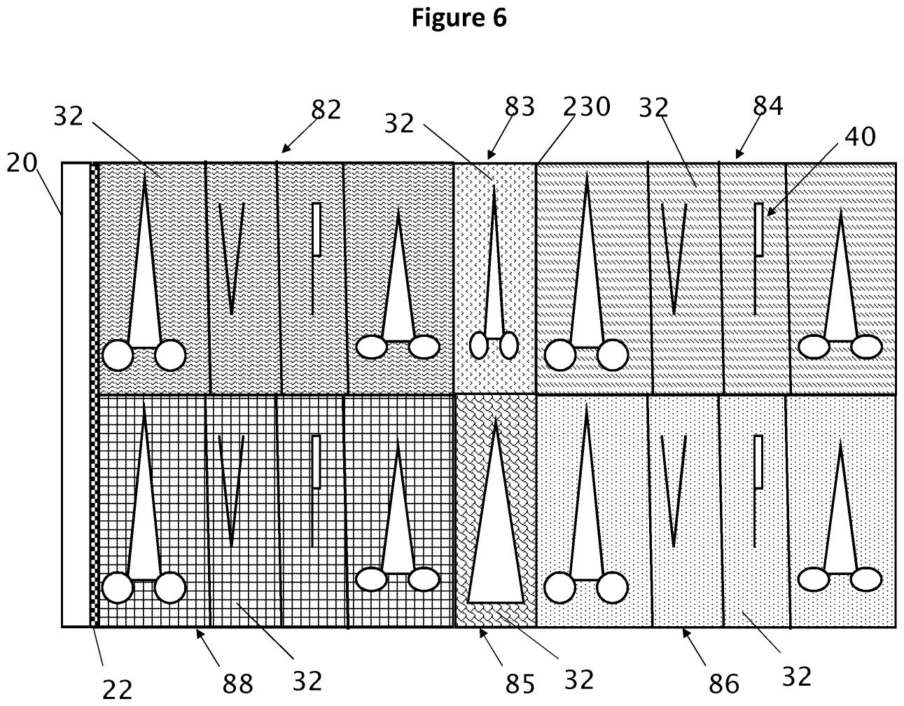

FIG. 6 shows a schematic plan view of a third embodiment of a container for use in the sealed pack according to the second embodiment of the invention.

A pack according to a first embodiment of the invention is indicated generally at 10 on FIGS. 1 and 2. Pack 10 includes a container 30, a container support crate 80, a container support lid 20, and seals 50,60,70. As shown in FIG. 2, container 30 is in the form of a pouch or bag which has a container aperture 28 such that the contents 40 of container 30 may be accessed. Container 30 has a container collar 35 around container aperture 28 and a tamper-evident transparent container seal 50 to cover and protect container aperture 28 whilst still allowing the contents 40 of container 30 to be inspected. In an alternative embodiment, container 30 may be formed from a transparent material and container seal 50 may be translucent or opaque.

As shown in FIG. 1, container support 80 is in the form of a crate formed from a cuboid lattice having sides which form openings 86 to allow inspection of the container 30, an open base and a container support top which is closed by container support lid 20. The sides 86 have edges which are defined by horizontal slats 82 and vertical slats 84. Container support top forms a container support aperture (not shown) which is defined by container support aperture slats (not shown) which are shaped such that container 30 may be inserted into container support 80 through the container support aperture. The container support aperture slats support container collar 35 such that container 30 is suspended in container support 80. The container support 80 is shaped such that container 30 is spaced from the sides 86 of support 80 such that support 80 protects container 30 and its contents. In an alternative embodiment, container support 80 may be in the form of a box having solid sides. In an alternative embodiment, the container support 80 may support a plurality of containers 30. In an alternative embodiment, the container support 80 may not have the container support aperture slats such that container support 80 can be used with one or more containers 30, each in the form of a tray or a box.

As shown in FIG. 1, container support lid 20 is formed from a resilient plastics material and has a corresponding shape to the container support top such that the lid 20 may be used to seal container 30. Lid 20 has a clip 26 for securing the closure 24 of lid 20 and container support 80. Lid 20 is connected to container support 80 by a hinge (not shown) such that the lid 20 may be moved in relation to the container support 80. The container support lid 20 has a lid clip seal to close and secure the lid on support 80. Lid 20 forms a recess shaped to receive the container seal 50 and the container collar 35 such that the closure 24 of lid 20 and container support 80 support container 30.

Seals 50,60,70 comprise an internal tamper-evident container seal 50, an external tamper-evident lid clip seal 60 and an external tamper-evident film seal 70. Furthermore, lid 20 provides an external tamper-proof seal. Container seal 50 is an internal seal as it is arranged internally in pack 10 between the container 30 and lid 20. Container seal 50 is in the form of a removable film seal and is for use by a consumer or recipient of the container 30 such that the consumer or recipient has proof that the contents of container 30 are secure. Outer film seal 70 is an external tamper-evident seal which is formed from a layer of plastics material which is wrapped around the pack 10 such that the lid 20 cannot be opened without removal of film seal 70 and is shown arranged on an external surface of the pack 10, in particular across closure 24 where lid 20 joins support 80. Lid clip seal 60 is a further external tamper-evident seal on the clip 26 which fastens lid 20 to container 30 and enables lid 20 to act as a further seal for container 30. The integrity of lid 20 and seals 60 and 70 provide reassurance during transit to transportation personnel that the support 80 is secure and that its contents have not been tampered with.

A pack according to a second embodiment of the invention is indicated generally at 110 on FIGS. 3, 4, and 5. Like features of the second embodiment of the pack 110 to the features of the first embodiment of the pack 10 are identified by like reference numerals. Pack 110 includes a container 130, a correspondingly shaped lid 20, a set of disposable sterilised surgical instruments 40 and seals 50,60,70.

As shown in FIGS. 4 and 5, container 130 is in the form of a tray and is formed from a resilient plastics material such that it protects and supports surgical instruments 40. Container 130 has an aperture 28 through which the surgical instruments 40 may be accessed. Aperture 28 of container 130 is sealed by internal transparent film seal 50. In an alternative embodiment, container 130 may be formed from a transparent resilient plastics material and internal film seal 50 may be opaque or translucent. In an alternative embodiment, container 130 may be used to protect and support food.

As shown in FIG. 1, lid 20 has a hinge 22 and a clip 26. Lid 20 is formed from a resilient transparent material such as a transparent plastics material and has a corresponding shape to the container 130 such that the lid 20 may be used to seal the contents of container 130. Lid 20 is connected to container 130 by hinge 22 such that the lid 20 may be moved in relation to the container 130 as shown in FIG. 2. Lid 20 may be secured to container 130 in its closed position by clip 26. In an alternative embodiment, for example where internal film seal 50 is opaque or translucent, lid 20 may be formed from an opaque or translucent material.

As shown in FIG. 1, seals 50,60,70 comprise an internal tamper-proof film seal 50, an external tamper-proof lid clip seal 60 and an external tamper-proof film seal 70. Outer film seal 70 is a tamper-evident seal which is arranged across closure 24 where lid 20 joins container 130. Lid clip seal 60 is a further tamper-evident seal on the clip 26 which fastens lid 20 to container 130 and enables lid 20 to act as a further seal for container 130. Inner film seal 50 is a tamper-evident seal provided across aperture 28 of container 130 such that a surgical instrument 40 may not be removed from the container 130 without removal of inner film seal 50.

As shown in FIGS. 4 and 5, the set of surgical instruments 40 includes surgical clamps 42, swab 43, scalpel 44, a first pair of surgical forceps 46 and a second pair of surgical forceps 48. Container 130 forms a plurality of tray compartments 32,34,36,38 which are shown in FIG. 4 or tray compartments 32,33,34,35,36,38 which are shown in FIG. 5. Each tray compartment 32,33,34,35,36,38 receives a respective surgical instrument 42,43,44,46,48 of the set of surgical instruments 40. In an alternative embodiment, each tray compartment 32,33,34,35,36,38 may be shaped to receive its respective surgical instrument 42,43,44,46,48. In an alternative embodiment where the respective surgical instrument 42,43,44,46,48 is formed from a suitable metal, its corresponding tray compartment 32,33,34,35,36,38 may have a magnet to help retain the respective surgical instrument 42,43,44,46,48 in its tray compartment 32,33,34,35,36,38. In an alternative embodiment, the plurality of tray compartments 32,34,36,38 of container 130 may be used to receive different types of food such as a different element of a meal such as food for a first, second or third course of a meal.

In an alternative embodiment, lid 20 may not have a hinge 22 and lid 20 may be mounted on container 130 such that it may be moved by sliding in relation to container 130 into an open position. In an alternative embodiment, the set of surgical instruments 40 may comprise the disposable sterilised surgical instruments 40 listed in Table 2A, 4, 6, 8, 10, 12A or 12B.

A container 230 according to a third embodiment of the invention for use with the pack according to the second embodiment of the invention is shown on FIG. 6. Container 230 forms a plurality of zones 82,83,84,85,86,88 of tray compartments 32 where the zones 82,83,84,85,86,88 are colour coded such that there are six colour coded groups of tray compartments 32 wherein each of which colour coded groups contains a group of surgical instruments having a pre-determined functionality as set out in Table 2B. For example for surgical instruments for use in a caesarean section, a first zone 82 may comprise tray compartments 32 and surgical instruments 40 in group 1 which are colour coded pink and may include surgical instruments 40 used to clean and prepare the skin for the intervention; a second zone 84 may comprise the tray compartments 32 and surgical instruments 40 in group 2 which are colour coded blue and may include surgical instruments 40 used to cut the skin (e.g. the abdomen); a third zone 83 may comprise the tray compartments 32 and surgical instruments 40 in group 3 which are colour coded yellow and may include the surgical instruments 40 that are used for handling tissue; a fourth zone 85 may comprise the tray compartments 32 and surgical instruments 40 in group 4 which are colour coded green and may include the surgical instrument that is used to deliver the baby; a fifth zone 86 may comprise tray compartments 32 and surgical instruments 40 in group 5 which are colour coded brown and may include the instruments that are used to close the uterus; and a sixth zone 88 may comprise the tray compartments 32 and surgical instruments 40 in group 6 which are colour coded red and may be used to close the abdomen and the skin.

TABLE 2A
Reduced surgical instrument set 40 for Caesarean section

| | Number of |
|---|---|
| Contents | instruments |
| Allis Tissue Forceps | |
| Green Armitage Forceps Curved | 0 |
| Green Armitage Forceps Straight | 2 |
| Criles Artery Forceps Curved | 0 |
| Spencer Wells Artery Forceps 7″ Straight | 2 |
| Kocher Artery Forceps 8″ Straight | 2 |
| Mayo Needleholder 7″ | 2 |
| Cord Scissors | |
| Dressing Scissors | 0 |
| Mayo Scissors Straight | 1 |
| Mayo Scissors Curved | 0 |
| McIndoe Scissors, Curved | 0 |
| Rampley Sponge Holding Forceps | 3 |
| Bonney Toothed Dissecting Forceps | 1 |
| Dissecting Forceps 7″ Non-Toothed | 1 |
| Waugh Dissecting Forceps Non-Toothed | 0 |
| Waugh Dissecting Forceps Toothed | 0 |
| Bard Parker Handle No. 4 | 2 |
| Wrigley Obstetric Forceps (Pair L & R) | 1 |
| Doyen Hand Retractor | 1 |
| Langenbeck Retractors Medium | 1 |
| Morris Retractors | 0 |
| Mono Polar Diathermy Cable | 1 |
| Towel Clip | 1 |
| Diathermy Dissecting Forceps 8″ | 1 |
| Chlorohexidine sprayer+++ | 1 |
| Shaver | 1 |
| Total | 25 |

TABLE 2B
Colour coded reduced surgical instrument
set 40 for Caesarean section

| | Number of instruments |
|---|---|
| | included in this tray |
| Pink tray for instruments | |
| used to clean the skin | |
| Shaver | 1 |
| Chlorohexidine sprayer+++ | 2 |
| Rampley Sponge Holding Forceps | 2 |
| Total | 5 |
| Blue tray for instruments | |
| used to open the abdomen | |
| Knife | 1 |
| Toothed | 1 |
| Scissors | 1 |
| Kochers | 2 |
| Total | 5 |
| Yellow tray for instruments | |
| used to handle the tissues | |
| Spencer wells | 1 |
| Non Toothed Forcep | 1 |
| Doyen | 1 Forceps |
| Total | 3 |
| Green tray for instruments | |
| used to deliver the baby | |
| Wrigleys | 1 |
| Brown tray for instruments | |
| used to close the uterus | |
| Needle holders | 2 |
| Toothed Forceps | 1 |
| Green Armitage | 2 |
| Total | 5 |
| Red tray for instruments | |
| used to close the abdomen | |
| Allis Tissue Forceps | 2 |
| Langenbeck Retractors Medium | 1 |
| Stapler | 1 |
| Total | 4 |

In an alternative embodiment, the set of surgical instruments 40 may comprise the reduced set of disposable sterilised surgical instruments listed in Table 4 for use in vaginal hysterectomy and repair. The full sets of surgical instruments for use in these interventions are listed in Tables 3A and 3B.

TABLE 3A
Full set of surgical instruments for Vaginal Hysterectomy

| | Number of instruments |
|---|---|
| Contents | included on the storage array |
| Forceps Rampley Sponge Holding | 5 |
| Bachaus Towel Clips | 5 |
| B.P. Handle No. 3 | 1 |
| B.P. Handle No. 4 | 2 |
| Scissors Dressing | 1 |
| Scissors Mayo Straight 5″ | 1 |
| Scissors Mayo Curved 5″ | 1 |
| Scissors McIndoes 7″ | 1 |
| Needleholder Mayo 7″ | 2 |
| Forceps Dissecting Bonney Toothed | 1 |
| Forceps Dissecting Non-Toothed 5″ | 1 |
| Forceps Dissecting Gillies Toothed | 1 |
| Forceps Artery Cairns Curved | 5 |
| Forceps Artery Kocher 6 | 10 |
| Forceps Artery Spencer Wells straight 9″ | 5 |
| Forceps Artery Kocher straight 8″ | 2 |
| Clamps Hysterectomy Gwilliams Curved | 4 |
| Forceps Tissue Allis | 4 |
| Forceps Vulsellum Teale | 2 |
| Dressing Forceps Bozeman Uterine | 1 |
| Bladder Sound | 1 |
| Female Catheter | 1 |
| Speculum Vaginal Medium | 1 |
| Retractor Vaginal Landon | 2 |
| Diathermy Lead | 1 |
| Diathermy Forceps 9″ | 1 |

TABLE 3B
Full set of surgical instruments for Vaginal/peritoneal Repair

| | Number of instruments |
|---|---|
| CONTENTS | included on the storage array |
| Mayo Scissors Straight | 1 |
| Mclndoe Scissors Curved | 1 |
| Rampley Sponge Holders | 2 |
| Crile Murray Needle Holder | 1 |
| Crile Artery Forceps Straight | 3 |
| Allis Tissue Forceps | 4 |
| Officer Rectal D/F, 4 × 5 toothed | 1 |
| Non-Tooted Dissecting Forceps | 1 |
| B.P. Handle No. 4 | 1 |
| London Vaginal Retractor | 1 |
| Sims Speculum Large D/E | 1 |

TABLE 4
Reduced instrument set for Vaginal hysterectomy and repair

| | Number of instruments |
|---|---|
| Contents | included on the storage array |
| Sims speculum | 2 |
| Vulsulum | 2 |
| Knife | 1 |
| Mayos scissors | 1 |
| Stitch scissors | 1 |
| Needle holders | 1 |
| Sponge forceps | 1 |
| Kochers | 1 |
| Ellis tissue forceps | 1 |
| Littlewoods | 1 |
| Catheters | 1 |
| Total | 13 |

In a further alternative embodiment, the set of surgical instruments may comprise the reduced set of disposable sterilised surgical instruments listed in Table 6 for use in abdominal surgery. The full set of surgical instruments for use in such an intervention are listed in Tables 5A and 5B.

TABLE 5A
The current abdominal hysterectomy set of instruments

| | |
|---|---|
| Forceps Sponge Holding Rampley | 6 |
| Towel Clips Bachaus | 6 |
| Scissors Mclndoe curved | 1 |
| Scissors Mayo Straight | 1 |
| Scissors Mayo Curved 5½″ | 1 |
| Scissors Boney Curved 7½″ | 1 |
| Mayo Needleholder 7″ | 2 |
| Forceps Artery Cairns curved | 5 |
| ForcepsArtery Birkett | 5 |
| Forceps Artery Spencer Wells straight 9″ | 5 |
| Forceps Artery Roberts | 2 |
| Forceps Artery Kocher 9″ | 4 |
| Clamps Hysterectomy Maingot | 2 |
| Clamps Hysterectomy Gwilliam Curved | 2 |
| Clamps Hysterectomy Gwilliam Straight | 2 |
| Forceps Teale Vulsellum | 2 |
| Forceps Tissue Lanes | 2 |
| Forceps Tissue Allis | 2 |
| Forceps Tissue Babcock | 2 |
| Forceps Dissecting Boney Toothed 7″ | 1 |
| Forceps Bickford DC | 1 |
| Forceps Dissecting Non-Toothed 6″ | 1 |
| Forceps Atraumatic Debakey 8″ | 1 |
| B.P. Handle No. 3 | 1 |
| B.P. Handle No. 4 | 2 |
| Forceps Diathermy 8″ | 1 |
| Forceps Diathermy 6″ | 1 |
| Diathermy Lead | 1 |
| Retractor Morris | 2 |
| Retractor Langenbeck Medium | 2 |
| Retractor Worral Bladder | 1 |
| Retractor Deaver (Medium) | 1 |
| Retractor Balfour Self-Retaining & 3^{rd} Blade | 1 |
| Myomectomy Screw | 1 |
| Poole Sucker with Fenestrated Guard | 1 |

TABLE 5B
The current laparotomy set of instruments

| | Current number of |
|---|---|
| Contents | instruments |
| Simms Speculum | 1 |
| Vulsellum Tissue Forceps | 2 |
| Curved Mayo Scissors | 1 |
| Set Hegar dialator 3-18 | 1 |
| Small Curette | 1 |
| Medium Curette | 1 |
| Large Curette | 1 |
| Polpectomy Forceps | 1 |
| Ovum Forceps | 1 |
| Rampley Sponge Forceps | 2 |
| Uterine sound | 1 |
| Bonney's Dissecting Forceps non-toothed | 1 |
| Medium Receiver | 1 |
| Blue Gallipot | 1 |
| 137 × 137 Inner Wrap | 1 |
| Transportation outer Wrap | 1 |
| Total | 18 |

TABLE 6
Reduced surgical instrument set 40 for
abdominal hysterectomy and laparotomy

| | Number of instruments |
|---|---|
| Contents | included on the storage array |
| Knife | 1 |
| Lange beck | 1 |
| Doyens retractor | 1 |
| Seif retaking retractor | 1 |
| Retractor | 1 |
| Bowel | 1 |
| Stitch Scissor | 1 |
| Mayos | 1 |
| Kochers | 2 |
| Maingot | 2 |
| Gwellim | 2 |
| Ellis | 4 |
| Spencer Wells | 4 |
| Total | 22 |

In a further alternative embodiment, the set of surgical instruments may comprise the reduced set of disposable sterilised surgical instruments listed in Table 8 for use in termination of pregnancy. The full set of surgical instruments for use in such an intervention is listed in Table 7.

TABLE 7
Surgical instrument set 40 for termination of pregnancy

| | Number of instruments |
|---|---|
| Contents | included on the storage array |
| Sponge Holders | 2 |
| Scissors Stitch | 1 |
| Forceps Ovum Mclintock | 1 |
| Forceps Polyp Bonney | 1 |
| Forceps Vulsellum Teals | 2 |
| Curette Evans | 4 |
| Curette Single Ended Sharp Small | 1 |
| Uterine Sound | 1 |
| Hawkins Ambler Dilators Set of 11 | 1 |
| (3 to 13) | |
| Sims Speculum | 1 |
| Syringe | 1 |
| Total | 16 |

TABLE 8
Reduced surgical instrument set 40 for termination of pregnancy

| | Number of instruments |
|---|---|
| Contents | included on the storage array |
| Sponge Holders | 2 |
| Forceps Polyp Bonney | 1 |
| Forceps Vulsellum Teals | 2 |
| Curette Evans | 1 |
| Curette Single Ended Sharp Small | 1 |
| Uterine Sound | 1 |
| Hawkins Ambler Dilators Set of 8 | 1 |
| Sims Speculum | 1 |
| Total | 16 |

In a further alternative embodiment, the set of surgical instruments may comprise the reduced set of disposable sterilised surgical instruments listed in Table 10 for use in tracheostomy surgery. The full set of surgical instruments for use in such an intervention are listed in Table 9.

TABLE 9
The current tracheostomy set of instruments

| Contents | No. |
|---|---|
| Retractor West self-retaining | 2 |
| Rampley sponge holding forceps | 2 |
| Bachaus towel clips | 5 |
| Lahey Forcep | 1 |
| Needleholder Stifles | 1 |
| Needleholder Gillies | 1 |
| Tissue Forceps Allis | 2 |
| Artery Forceps Cairns, curved | 5 |
| Artery Forceps Halstead mosquito, curved | 5 |
| Scissors Iris, sharp, straight | 1 |
| Scissors Strabismus, blunt, straight | 1 |
| Scissors Strabismus, blunt, curved | 1 |
| Scissors McIndoe, curved | 1 |
| Tracheal dilator | 1 |
| Dissecting forceps McIndoe, non-toothed | 1 |
| Dissecting forceps Gillies, toothed | 1 |
| Magil sucker no 3 | 2 |
| Skin hook Gillies | 2 |
| Skin hook Kilner | 1 |
| Retractor Kilner catspaw | 2 |
| Retractor double hook, blunt | 2 |
| Retractor single hook, blunt | 1 |
| Cricoid hook, sharp | 1 |
| Probe Dissector Watson Cheyne | 1 |
| Bard parker handle no 3 | 1 |
| Bard parker handle no 4 | 1 |
| Retractors Langenbeck, small & medium | 2 ea |
| Total | 72 |

TABLE 10
Reduced surgical instrument set 40 for tracheostomy

| | Number of instruments |
|---|---|
| Contents | included on the storage array |
| Surgical knife | 1 |
| Tooth forceps | 1 |
| Dissecting scissor | 1 |
| Surgical retractors | 1 |
| Cricoid hook | 1 |
| Bipolar diathermy | 1 |
| Cuffed and non-cuffed tracheostomy tubes | 1 |
| Dressings | 1 |
| Total | 8 |

In a further alternative embodiment, the set of surgical instruments may comprise the reduced set of disposable sterilised surgical instruments listed in Table 12A or 12B for use in tonsil and adenoid surgery. The full set of surgical instruments for use in such an intervention are listed in Table 11.

TABLE 11
The current Tonsil & adenoids set of instruments

| | Current number of |
|---|---|
| Contents | instruments |
| Negus Tonsil Artery Forceps, large curve | 1 |
| Negus Tonsil Artery Forceps, small curve | 1 |
| Birkett Straight Artery Forceps | 1 |
| Birkett Curved Artery Forceps | 1 |
| Denis Browne Tonsil Holding Forceps | 1 |
| Luc's Tonsil Holding Forceps | 1 |
| McIndoes Scissors | 1 |
| Wilson Scissors | 1 |
| Mollisons Tonsil Dissector/retractor | 1 |
| Gwynne Evans Tonsil Dissector | 1 |
| Negus Ligature pusher | 1 |
| Adenoid Curette & spiked cage | 1 |
| Adenoid Curette | 4 |
| Waughs Dissecting Forceps Non-toothed | 1 |
| Waughs Dissecting Forceps Toothed | 1 |
| Boyle Davis Gag | 1 |
| Split blade, varying sizes | 4 |
| Draffin bipods | 2 |
| Yankauer Sucker | 1 |
| Towel Clip ball and socket | 2 |
| Bi-polar forceps & Lead | 1 ea |
| Total | 18 |

TABLE 12A
First reduced surgical instrument set 40
for use in tonsil and adenoid surgery

| | Number of instruments |
|---|---|
| Contents | included on the storage array |
| Boyle Davis mouth gag | 1 |
| Tongue depressor | 1 |
| St Clair Ad curette | 1 |
| Denis Brown Ts holding forceps | 1 |
| Negus curved forceps | 1 |
| Berkett forceps | 1 |
| Knot pusher | 1 |
| Pillar retractors | 1 |
| Scissors | 1 |
| Total | 9 |

TABLE 12B
Second reduced surgical instrument set 40
for use in tonsil and adenoid surgery

| | Number of instruments |
|---|---|
| Contents | included on the storage array |
| Bipolar dissection for Tonsellectomy | 1 |
| Suction diathermy | 1 |
| Gag and tongue depressor | 1 |
| Tonsellectomy holder | 1 |
| Yanker suction | 1 |
| Total | 5 |

1. A sealed pack which pack comprises a container and a lid wherein the container has an internal tamper-evident seal and the lid has an external tamper-evident seal; wherein the internal seal is arranged between the container and the lid; and wherein the lid is formed from a resilient material such that it protects the container.

2. The pack as defined in claim 1 wherein the container is a tray, bag or pouch.

3. The pack as defined in claim 1 which comprises a support for protecting the container.

4. The pack as defined in claim 1 wherein the lid, container and/or the internal tamper-evident seal are transparent.

5. The pack as defined in claim 1 wherein the lid has a clip to secure the lid to the container.

6. The pack as defined in claim 5 wherein the external tamper-evident seal is provided on the clip.

7. The pack as defined in claim 1 wherein the external tamper-evident seal comprises a film seal.

8. The pack as defined in claim 1 which comprises one or more sterilised surgical instruments suitable for use in a pre-determined surgical intervention wherein the container is a tray for receiving the one or more sterilised surgical instruments.

9. The pack as defined in claim 8 wherein the one or more surgical instruments are disposable surgical instruments.

10. The pack as defined in claim 9 wherein, the one or more surgical instruments are formed from a plastic and/or metal material.

11. The pack as defined in claim 8 wherein the tray has one or more tray compartments for receiving the one or more surgical instruments.

12. The pack as defined in claim 11 wherein, the tray compartments are in the form of grooves or slots.

13. The pack as defined in claim 11 wherein, the one or more tray compartments are colour coded.

14. The pack as defined in claim 1 wherein, the tray is formed from a disposable, resilient and sterilisable material.

15. The pack as defined in claim 8 which includes the majority of surgical instruments required for the pre-determined surgical intervention

16. The pack as defined in claim 15 wherein, the pack contains from 50% to 90% of the required surgical instruments.

17. The pack as defined in claim 8 which includes one or more checklists which lists the names of the one or more surgical instruments in the pack.

18. The pack as defined in claim 1 wherein at least one of the seals is transparent.

19. A sealed pack which pack comprises a container and a lid wherein the container has an internal tamper-evident seal and the lid has an external tamper-evident seal; wherein the internal seal is arranged between the container and the lid; wherein the internal seal is transparent and wherein the lid is formed from a transparent resilient material such that the lid protects the container.

20. A sealed pack which pack comprises a container and a lid wherein the container has an internal tamper-evident seal and the lid has an external tamper-evident seal; wherein the internal seal is arranged between the container and the lid; wherein the lid is formed from a resilient material such that it protects the container; wherein the pack comprises one or more sterilised surgical instruments suitable for use in a pre-determined surgical intervention; and wherein the container is a tray for receiving the one or more sterilised surgical instruments.
